(19) United States
(12) Patent Application Publication
(10) Pub. No.: US 2016/0251690 A1
(43) Pub. Date: Sep. 1, 2016

(54) METHOD FOR TREATING CELLULOSE-CONTAINING BIOMASS

(71) Applicant: SHOWA DENKO K.K., Tokyo (JP)

(72) Inventors: Ichiro FUJITA, Tokyo (JP); Shinji YAMAKI, Tokyo (JP); Tadashi YONEDA, Tokyo (JP)

(73) Assignee: SHOWA DENKO K.K., Tokyo (JP)

(21) Appl. No.: 15/027,617

(22) PCT Filed: Sep. 9, 2014
(86) PCT No.: PCT/JP2014/073796

§ 371 Date: Apr. 6, 2016

(30) Foreign Application Priority Data
Oct. 7, 2013 (JP) 2013-210212

(51) Int. Cl. C12P 19/14; C12P 19/02

(52) U.S. Cl. CPC C12P 19/14; C12P 19/02; C12P 2201/00

(57) ABSTRACT

A treatment method for biomass, which is a pretreatment method including continuously performing hydrothermal treatment using a screw extruder to produce a biomass composition for saccharification from cellulose-containing biomass and containing only a small quantity of furfural, the treatment method sequentially including: pulverizing the cellulose-containing biomass so as to have a maximum grain size of 1,000 μm or less and adjusting a water content ratio thereof to from 30% to 80%; performing hydrothermal treatment in which an element including a seal ring and at least one set of a kneading disc and/or a left-hand screw is arranged immediately upstream of the seal ring, at a temperature of (1) from 150° C. to 200° C. or (2) from 200° C. to 215° C. for 0.1 minute to 15 minutes while performing kneading pulverization having a grinding effect; and cooling a treated product to 100° C. or less to recover the treated product.

### TECHNICAL FIELD

The present invention relates to a treatment method for cellulose-containing biomass. More specifically, the present invention relates to a treatment method for cellulose-containing biomass involving continuously performing hydrothermal treatment through use of a screw extruder to produce a biomass composition exhibiting high saccharification performance from the cellulose-containing biomass serving as a raw material while suppressing the generation of a furfural by-product as being a substance inhibiting fermentation; a production method for a biomass composition for saccharification; and a production method for a sugar.

### BACKGROUND ART

As part of measures against global warming, there have been wide investigations on production of various chemical products including ethanol and the like through effective utilization of cellulose-containing biomass. Examples of the cellulose-containing biomass include hard biomass such as cedar or cypress, and soft biomass, such as rice straw, wheat straw, corncobs, cassava, bagasse, or sugar cane leaves. The biomass may contain hemicellulose, lignin, and the like, and hence it is difficult to directly saccharify the biomass. Therefore, there have been proposals to enhance its saccharification performance through various pretreatments.

As a pretreatment method for enhancing the saccharification performance, there have been proposed a method involving adding an acid or alkali and performing hydrothermal treatment, and a method involving a combination of hydrothermal treatment without a chemical and physical pulverization treatment (JP 2006-136263 A; Patent Document 1). Further, in addition to those methods, there have been proposed water vapor blasting, ammonia blasting, ozone oxidation, white-rot fungus treatment, microwave irradiation, electron beam irradiation, and γ-ray irradiation (Journal of The Japan Wood Research Society, 53, 1-13 (2007); Non-Patent Document 1).

However, when those pretreatment methods are studied for industrially useful treatment steps, a device and a method capable of continuously and efficiently treating a raw material in a large amount are not specifically disclosed, while those methods each have an effect of enhancing the saccharification performance to some extent.

As a method of continuously and efficiently performing pretreatment prior to enzymatic saccharification of biomass, in JP 59-192093 A (Patent Document 2), and JP 59-192094 A and JP 2012-170355 A (Patent Documents 3 and 4; U.S. Pat. No. 4,642,287), it has been proposed that a pretreatment method involving kneading biomass with an alkali and subjecting the biomass to hydrothermal treatment with a twin-screw extruder can be continuously performed at a high concentration in a short treatment time as compared to conventional fine pulverization treatment and alkali steam treatment.

However, the pretreatment methods disclosed in Patent Documents 2 to 4 each involve high chemical cost owing to the use of the alkali at a ratio of around 20% with respect to a raw material, and inevitably require neutralization and washing of the added alkali prior to the enzymatic saccharification. Therefore, a problem in terms of economy and efficiency including even a saccharification step is not solved. Further, the pretreatment methods of Patent Documents 2 and 3 are each disclosed as substantially a combination of pulverization and alkali steaming, but with regard to the treatment conditions, only the conditions related to the alkali steaming, such as a heating temperature, a heating time, and the amounts of the raw material and the alkali to be loaded, are presented, and the configuration of a device related to the pulverization, etc. are not presented. The embodiments for carrying out those inventions are unclear.

As a method of easily and rapidly performing pretreatment of plant biomass, in JP 2011-130745 A (Patent Document 5), there has been proposed a method involving sequentially performing pretreatment operations in an extruder in a continuous manner, the operations involving adding a decomposer to plant biomass coarsely pulverized into a preset size or less and subjecting the plant biomass to pressurized hot water treatment, and subsequent operations before saccharification loading in which the plant biomass is mixed with an enzyme for saccharification. However, in Patent Document 5, the conditions under which the treatment method is performed, and data on saccharification of the biomass as to what level of saccharification performance is obtained are not presented, while a flowchart and a screw configuration of the extruder for the method are disclosed. The overall performance and efficiency including even a saccharification step are unclear.

In view of the foregoing, there is a demand for establishment of a pretreatment method for cellulose-containing biomass which is capable of continuously treating a raw material in a large amount to thereby balance obtaining high saccharification performance and obtaining a sugar of practical use containing only a small quantity of a fermentation-inhibiting component, and industrially useful.

### PRIOR ART

### Patent Document

### Patent Document 1: JP 2006-136263 A

Patent Document 2: JP 59-192093 A (U.S. Pat. No. 4,642,287)
Patent Document 3: JP 59-192094 A (U.S. Pat. No. 4,642,287)

### Patent Document 4: JP 2012-170355 A

### Patent Document 5: JP 2011-130745 A

### Non-Patent Document

### Non-Patent Document 1: Journal of the Japan Wood Research Society, 53, 1-13 (2007)

### DISCLOSURE OF INVENTION

### Problem to be Solved by Invention

An object of the present invention is to provide a pretreatment method for cellulose-containing biomass which is capable of continuously treating a raw material in a large amount to thereby balance obtaining high saccharification performance and obtaining a sugar of practical use containing only a small quantity of a fermentation-inhibiting component, and industrially useful; a production method for a cellulose-containing composition for saccharification including conducting the treatment method; and a production method for a sugar including hydrolyzing the cellulose-containing composition for saccharification.

### Means to Solve Problem

The inventors of the present invention have made extensive investigations in order to achieve the above-mentioned object. As a result, the inventors have found that, when, in a treatment method for obtaining a sugar from cellulose-containing biomass, a screw extruder is used, and the cellulose-containing biomass is sequentially subjected to: pulverization and adjustment of a water content ratio in a pulverization section of the device; hydrothermal treatment concurrently with kneading pulverization having a grinding effect in a heating section of the device; and cooling in a cooling section of the device, in a continuous manner, a cellulose-containing composition exhibiting high saccharification performance to glucose, in which generation of furfural as being a fermentation-inhibiting component is suppressed, is obtained. Thus, the present invention has been completed.

That is, the present invention relates to the following treatment method for cellulose-containing biomass.

[1] A treatment method for biomass, which is a pretreatment method comprising continuously performing hydrothermal treatment through use of a screw extruder to produce from cellulose-containing biomass serving as a raw material a biomass composition for saccharification containing only a small quantity of furfural as being a fermentation-inhibiting component,
the treatment method sequentially comprising:
pulverizing, in a pulverization section of the screw extruder, the cellulose-containing biomass serving as a raw material so as to have a maximum grain size of 1,000 μm or less and adjusting a water content ratio thereof to from 30% to 80%;
performing hydrothermal treatment in a heating section of the screw extruder, in which an element including a seal ring and at least one set of a kneading disc and/or a left-hand screw is arranged immediately upstream of the seal ring, at a temperature of from 150° C. to 200° C. for 0.1 minute to 15 minutes while performing kneading pulverization having a grinding effect; and
cooling, in a cooling section of the screw extruder downstream of the heating section, a treated product to 100° C. or less to recover the treated product of the cellulose-containing biomass.
[2] A treatment method for biomass, which is a pretreatment method comprising continuously performing hydrothermal treatment through use of a screw extruder to produce from cellulose-containing biomass serving as a raw material a biomass composition for saccharification containing only a small quantity of furfural as being a fermentation-inhibiting component,
the treatment method sequentially comprising:
pulverizing, in a pulverization section of the screw extruder, the cellulose-containing biomass serving as a raw material so as to have a maximum grain size of 1,000 μm or less and adjusting a water content ratio thereof to from 30% to 80%;
performing hydrothermal treatment in a heating section of the screw extruder, in which an element including a seal ring and at least one set of a kneading disc and/or a left-hand screw is arranged immediately upstream of the seal ring, at a temperature of from 200° C. to 215° C. for 0.1 minute to 2.0 minutes while performing kneading pulverization having a grinding effect; and
cooling, in a cooling section of the screw extruder downstream of the heating section, a treated product to 100° C. or less to recover the treated product of the cellulose-containing biomass.
[3] The treatment method for biomass according to [1] or [2] above, in which, in the cooling section downstream of the heating section, the cooling is performed by installing a water cooling jacket and/or a liquid feed line.
[4] The treatment method for biomass according to any one of [1] to [3] above, in which the screw extruder is a co-rotating twin-screw extruder.
[5] The treatment method for biomass according to any one of [1] to [4] above, in which the cellulose-containing biomass is soft biomass.
[6] A production method for a biomass composition for saccharification, comprising conducting the treatment method described in any one of [1] to [5] above.
[7] A production method for a sugar, comprising hydrolyzing a biomass composition obtained by the production method described in [6] above.

### Effects of Invention

According to the treatment method for cellulose-containing biomass of the present invention comprising continuously performing the hydrothermal treatment concurrently with the kneading pulverization having a grinding effect of a cellulose-containing biomass through use of a screw extruder, a cellulose-containing composition exhibiting high saccharification performance to glucose, in which generation of furfural as being a fermentation-inhibiting component is suppressed, is obtained. Accordingly, a sugar can be produced from the biomass by balancing quality and productivity.

### BRIEF DESCRIPTION OF DRAWINGS

FIG. 1(A) is a configuration view of a screw extruder A used in Example 2, FIG. 1(B) is a configuration view of a screw extruder B used in Example 1, FIG. 1(C) is a configuration view of a screw extruder C used in Examples 3 and 4, and Comparative Examples 3 and 4, FIG. 1(D) is a configuration view of a screw extruder D used in Comparative Example 2, and FIG. 1(E) is a configuration view of a screw extruder E used in Comparative Example 1.

### MODE FOR CARRYING OUT INVENTION

The present invention is described in detail below. A treatment method of the present invention comprises feeding cellulose-containing biomass to a screw extruder, and continuously performing hydrothermal treatment concurrently with kneading pulverization having a grinding effect. An acid or alkali as an additive may be added to water in a raw material used in the treatment method, but it is industrially preferred to use only water, which is generally available, because the use of the additive not only increases chemical cost but also produces cost for detoxification, such as neutralization, in a subsequent step.

### Cellulose-Containing Biomass

The biomass used in the treatment method of the present invention means a biopolymer (nucleic acid, protein, or polysaccharide) or an industrial resource derived from such constituent component, other than exhaustible resources (fossil fuel, such as petroleum, coal, or natural gas). Therefore, examples of the cellulose-containing biomass include hard biomass such as wood, and soft biomass such as rice straw, wheat straw, corncobs, cassava, bagasse, or sugar cane leaves. Soft biomass is preferred in consideration of the ease of the pretreatment, and further, bagasse and sugar cane leaves are particularly preferred in consideration of their global storage potential and collection cost.

### Type of Screw Extruder

The screw extruder to be used in the treatment method of the present invention may be any one of a single-screw extruder, a multi-screw extruder, and a special extruder. Of those, a multi-screw extruder, which can apply stronger shear to a biomass material, is preferred, and a twin-screw extruder is more preferred because of its generality and versatility.

As the multi-screw extruder, there may be adopted any one of a type in which screw shafts are parallel to one another and a conical type in which the screw shafts cross obliquely one another. Of those, a parallel type is preferred.

Any one of an engaged-screw type and a non-engaged-screw type may be adopted, but of those, an engaged-screw type is preferred because of a high kneading effect and many practical examples.

With regard to a screw rotation direction, any one of a co-rotation type and a counter-rotation type may be adopted, but of those, a co-rotation type is preferred because of a self-cleaning effect.

### Raw Material Feed Portion of Screw Extruder

A hopper to be used to stably feed a raw material to a cylinder of the screw extruder is not limited as long as the hopper has a function capable of generating a feed pressure required for a feed portion of a screw without causing bridging of the raw material, and examples of such hopper include a vibration hopper, a hopper with a force feeder, a hopper dryer, a vacuum hopper, and a nitrogen purge hopper. A hopper comprising a screw located on the inside of the hopper and configured to forcibly push a material into the cylinder is preferred from the viewpoint of stably feeding the raw material.

A device configured to quantitatively feed the raw material to the screw extruder is mounted below the hopper. The quantitative feeding device is not limited as long as the device has a function of enabling quantitative feeding, and examples of such device include a mass feeder and a constant volume feeder. Of those, a mass feeder is preferred in view of feeding raw material biomass, which generally has a low bulk density and non-uniform shapes and sizes. In order to feed the raw material to the screw extruder more securely, it is preferred to mount a compactor configured to forcibly press the raw material into the extruder through use of a screw or a piston so that the bulk density of the material can be increased.

### Cylinder Portion of Screw Extruder

A cylinder portion of the screw extruder includes the following three sections: a heating section, which is located in the middle portion of the cylinder, and is configured to perform hydrothermal treatment through heating with a heater while grinding the raw material; a pulverization section, which is located upstream of the heating section, and is configured to pulverize the raw material and adjust its water content ratio, to thereby consolidate the material and maintain airtightness; and a cooling section, which is located downstream of the heating section, and is configured to cool the material, to thereby consolidate the material and maintain the airtightness. The screw extruder has an L/D of preferably from 30 to 80, more preferably from 40 to 80, still more preferably from 50 to 80 in its entirety including the pulverization section, the heating section, and the cooling section from the viewpoint of stably maintaining sealing, and performing hydrothermal treatment having an effect of improving the saccharification performance of the raw material biomass. The pulverization section has an L/D of preferably from 10 to 40, more preferably from 10 to 30, still more preferably from 15 to 25. In addition, the heating section has an L/D of preferably from 10 to 65, more preferably from 15 to 60, still more preferably from 20 to 55. The cooling section has an L/D of preferably from 5 to 35, more preferably from 5 to 20, still more preferably from 5 to 10. It should be noted that the “L/D” refers to an effective length represented by a ratio between the length (L) of a screw, which is measured from a start point of screw thread below the hopper to the tip of the screw, and the diameter (D) of the screw.

### Configuration of Pulverization Section of Cylinder

The pulverization section of the cylinder preferably has a screw configuration in which at least one or more elements each comprising a seal ring and at least one set of a kneading disc (feed kneading disc, neutral kneading disc, or reverse kneading disc) or a left-hand screw arranged upstream of the seal ring (hereinafter abbreviated as “seal ring elements”) are arranged. A state in which the raw material on an upstream side is compressed is achieved by a damming effect exhibited by the arranged seal ring, thereby achieving a state in which the shear force of a screw located upstream of the seal ring is increased. As a result, the raw material is efficiently pulverized and consolidated, and exhibits a function of sealing the pressure of vapor to be generated in the heating section. The pulverization of the raw material is performed not only for achieving the sealing function but also for improving efficiency of the hydrothermal treatment in the heating section. For this, the maximum grain size of the raw material is preferably set to 1,000 μm or less. It should be noted that the maximum grain size is determined through microscopic observation of a sample extracted from the pulverization section immediately upstream of the heating section.

In addition, in the pulverization section, the water content ratio of the raw material is adjusted to preferably from 30 mass % to 80 mass %, more preferably from 30 mass % to 75 mass %, still more preferably from 35 mass % to 70 mass % in order to optimally perform a hydrothermal reaction and achieve an optimal sealing property. The adjustment of the water content ratio may be separately performed before loading, but is preferably performed by installing a liquid feed line in an arbitrary portion of the pulverization section to feed water therethrough with a high-pressure pump from the viewpoint of reducing the number of steps. It should be noted that the water content ratio refers to the ratio of the mass of water to the total mass of the raw material as it is.

### Configuration of Heating Section of Cylinder

The heating section of the cylinder preferably has a screw configuration in which at least three or more sets of seal ring elements are arranged. The arrangement of a plurality of seal rings in the heating section exhibits such effect that strong grinding stress, which is generated when the cellulose-containing biomass serving as a raw material passes through an extremely narrow clearance between the seal ring and the cylinder, is applied concurrently with the hydrothermal treatment, thereby improving the saccharification performance of cellulose in biomass. For such effect, the clearance between the seal ring and the cylinder is preferably from 0.5% to 10.0%, more preferably from 1.0% to 8.0%, still more preferably from 1.5% to 5.0% with respect to the inner diameter of the cylinder. It should be noted that the inner diameter of the cylinder in a twin-screw extruder refers to the diameter of a circle surrounding one screw in a vertical cross section of the cylinder. The heating in the heating section is not limited as long as the cylinder can be heated, but is preferably performed with an electric heater from the viewpoint of temperature controllability. As the conditions of the hydrothermal treatment for the raw material, the hydrothermal treatment temperature and the time of passage through the heating section are preferably from 150° C. to 200° C. for 0.1 to 15 minutes or from 200° C. to 215° C. for 0.1 to 2.0 minutes, more preferably from 160° C. to 200° C. for 1 to 10 minutes or from 200° C. to 213° C. for 0.3 to 2.0 minutes, still more preferably from 160° C. to 200° C. for 2 to 8 minutes or from 200° C. to 210° C. for 0.5 to 1.5 minutes. A pressure in the heating section falls within a range of preferably from 1 MPa to 20 MPa, more preferably from 1 MPa to 15 MPa, still more preferably from 2 MPa to 12 MPa.

### Configuration of Cooling Section of Cylinder

The cooling section of the cylinder preferably includes a water cooling jacket and/or a liquid feed line in order to cool the raw material heated in the heating section. The cooling in the cooling section is performed so that the temperature of the raw material is reduced to preferably 100° C. or less, more preferably 80° C. or less, still more preferably 70° C. or less. With this, vapor generated in the heating section turns into water, and the pressure of vapor flowing downstream together with the treated biomass can be sealed. Further, a pressure regulating valve may be mounted to a discharge port in the cooling section in order to seal the the pressure of vapor more stably in the system.

### Pulverization (Adjustment of Grain Size)

In the treatment method of the present invention, the cellulose-containing biomass serving as a raw material may be directly fed to the screw extruder without pulverization treatment, but is preferably subjected to adjustment of a grain size in advance through coarse pulverization before its feeding. Pulverization means is not particularly limited as long as the means has a function capable of pulverizing a solid substance. For example, the mode of the device may be a dry mode or a wet mode. In addition, the pulverization system of the device may be a batch system or a continuous system. Further, the pulverization force of the device may be provided by any of impact, compression, shearing, friction, and the like.

Preliminary pulverization treatment may be performed with a device which may be used for the pulverization treatment. Specific examples of the device include a coarse crusher, such as a shredder, a jaw crusher, a gyratory crusher, a cutter mill, a cone crusher, a hammer crusher, a roll crusher, or a roll mill; or a medium crusher, such as a stamp mill, an edge runner, a cutting/shearing mill, a rod mill, an autogenous mill, or a roller mill. Of those, a cutter mill is preferred from the viewpoints of a treatment amount and a pulverization range. The time for treating the raw material is not particularly limited as long as the raw material can be homogeneously and finely pulverized by the treatment.

The grain size of the raw material subjected to pulverization in advance before its feeding is preferably a size passing through a screen (sieve) having a screen diameter of from 0.5 mm to 30 mm because, when the discharge screen diameter of the pulverization device is excessively large, the grain size of the cellulose-containing biomass increases, resulting in high sugar production cost owing to a reduction in subsequent pretreatment effects, and when the screen diameter is excessively small, pulverization cost increases. The grain size is more preferably a size passing through a screen of from 1 mm to 30 mm, most preferably a size passing through a screen of from 3 mm to 30 mm. In addition, also in the case of performing pulverization without using a screen, it is preferred to pulverize the raw material so as to achieve a size corresponding to that of a pulverized product in the case of using the screen.

### Adjustment of Water Content Ratio

In the treatment method of the present invention, the cellulose-containing biomass serving as a raw material may be subjected to adjustment of a water content ratio in advance before its feeding to the screw extruder. As a method of adjusting a water content ratio, there are given addition of water, dewatering, and drying, in accordance with the water content ratio of the raw material before the adjustment. As described above, the water content ratio of the raw material is preferably adjusted to from 30 mass % to 80 mass % in order to optimally perform the hydrothermal reaction and achieve an optimal sealing property.

### Saccharification of the Hydrothermally Treated Product

A biomass composition for saccharification can be efficiently produced by treating the biomass by the above-mentioned method. Further, when the biomass composition for saccharification produced by the above-mentioned method is hydrolyzed, a sugar can be efficiently produced.

The saccharification treatment of the cellulose-containing biomass subjected to hydrothermal treatment can be conducted by, after a dewatering process by solid-liquid separation before saccharification or after washing out insoluble components by adding water, adjusting pH and adding an enzyme. When the concentration of furfural as a by-product of the hydrothermal treatment is low, an effect of skipping the process of washing the insoluble components or reducing the load in the process can be obtained. For the solid-liquid separation before saccharification, a device such as a belt filter, a centrifugal filter, a press filter, an Oliver filter and a centrifuge can be used. It is preferable to conduct the separation with a belt filter from the viewpoint that biomass can be continuously treated in a large amount.

As another way of saccharification, saccharification treatment can be conducted by directly adjusting pH of the treated product and adding an enzyme thereto. In this case, the concentration of furfural as a by-product needs to be suppressed to a level such that fermentation-inhibition may not be directly caused by furfural because there is no washing process after the hydrothermal treatment.

### EXAMPLES

The present invention is hereinafter described by way of Examples and Comparative Examples. However, the present invention is by no means limited to the descriptions of Examples and Comparative Examples.

In Examples 1 to 4 and Comparative Examples 1 to 4, screw extruders under five kinds of device conditions were used, and cellulose-containing biomass was treated with changing the treatment conditions (a hydrothermal temperature, a hydrothermal time, a screw configuration, a number of sealing elements in the heating section, a rotation number of the screw, a feed rate of a mass of the raw material as it is, a feed rate of the raw material in terms of dry mass, and a feed rate of water) as shown in Table 1. The saccharification reaction of the treated sample was performed to confirm the generated sugar and the furfural concentration. Further, the obtained sugar solution was cultured for evaluation.

### Screw Extruders A to E

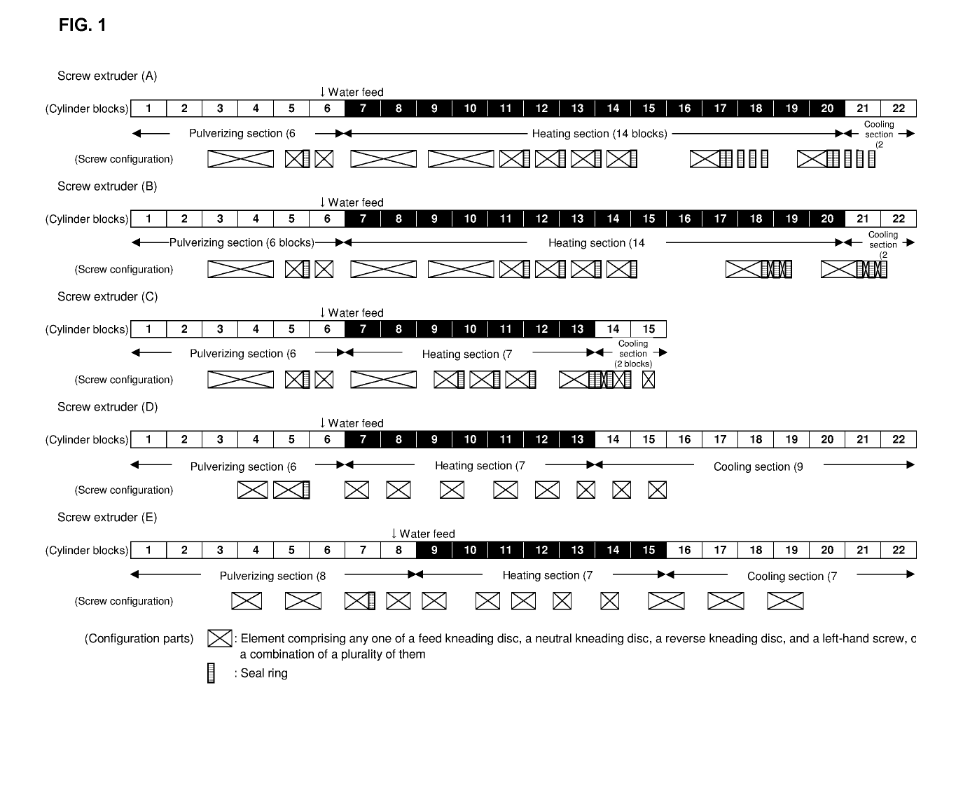

A twin-screw extruder having a screw diameter of 32 mm (trade name: TEX30α, manufactured by The Japan Steel Works, Ltd.) or a twin-screw extruder having a screw diameter of 47 mm (trade name: TEX44α, manufactured by The Japan Steel Works, Ltd.) was used in the experiment. The extruders was set to have any of the screw configurations A to E (illustrated in FIG. 1(A) to FIG. 1(E)) and used in Examples and Comparative Examples shown in Table 1.

TABLE 1

| | | | | | | | | | Number of seal rings | | | |
|---|---|---|---|---|---|---|---|---|---|---|---|---|
| | L/D | | | | Number of blocks | | | | used | | | |
| Extruder | Whole | Pulverization | Heating | Cooling | Pulverization | Heating | Cooling | | Pulverization | Heating | Cooling | |
| No. | device | section | section | section | section | section | section | Total | section | section | section | Total |
| Screw | 77 | 21 | 49 | 7 | 6 | 14 | 2 | 22 | 1 | 11 | 3 | 15 |
| extruder A | | | | | | | | | | | | |
| Screw | 77 | 21 | 49 | 7 | 6 | 14 | 2 | 22 | 1 | 7 | 3 | 11 |
| extruder B | | | | | | | | | | | | |
| Screw | 52.5 | 21 | 24.5 | 7 | 6 | 7 | 2 | 15 | 1 | 4 | 3 | 8 |
| extruder C | | | | | | | | | | | | |
| Screw | 77 | 21 | 24.5 | 31.5 | 6 | 7 | 9 | 22 | 1 | 0 | 0 | 1 |
| extruder D | | | | | | | | | | | | |
| Screw | 77 | 28 | 24.5 | 24.5 | 8 | 7 | 7 | 22 | 1 | 0 | 0 | 1 |
| extruder E | | | | | | | | | | | | |

| | | | | | Clearance | | | |
|---|---|---|---|---|---|---|---|---|
| | Number of seal ring | | | | between seal | | Diameter | |
| | elements | | | | ring and screw | | of | Examples or |
| Extruder | Pulverization | Heating | Cooling | | Length | Ratio | cylinder | Comparative |
| No. | section | section | section | Total | (mm) | (%) | (mm) | Examples used |
| Screw | 1 | 6 | 0 | 7 | 0.5 | 1.6 | φ32 | Example 2 |
| extruder A | | | | | | | | |
| Screw | 1 | 5 | 1 | 7 | 0.5 | 1.6 | φ32 | Example 1 |
| extruder B | | | | | | | | |
| Screw | 1 | 4 | 3 | 8 | 2.0 | 4.3 | φ47 | Examples 3, 4 |
| extruder C | | | | | | | | Comparative |
| | | | | | | | | Examples 3, 4 |
| Screw | 1 | 0 | 0 | 1 | 0.5 | 1.6 | φ32 | Comparative |
| extruder D | | | | | | | | Example 2 |
| Screw | 1 | 0 | 0 | 1 | 0.5 | 1.6 | φ32 | Comparative |
| extruder E | | | | | | | | Example 1 |

### Preparation of Raw Material Bagasse

Bagasse was used as the cellulose-containing biomass serving as a raw material.

Bagasse subjected to no treatment (water content ratio: 50%, content ratio of cellulose: 38%, hereinafter abbreviated as “untreated bagasse”), and bagasse (water content ratio: 10.0%, content ratio of cellulose: 42%, hereinafter abbreviated as “3-mm bagasse”) prepared by pulverizing air-dried bagasse with a cutter mill having a screen diameter of 3 mm (MKCM-3, manufactured by Masuko Sangyo Co., Ltd.) were used as the bagasse.

### Analysis Method for Content Ratios of Main Components of Biomass

The content ratio of cellulose, the content ratio of hemicellulose, and the total content ratio of lignin and an ash content in the biomass were determined by an analysis method (Technical Report NREL/TP-510-42618) of The National Renewable Energy Laboratory (NREL; U.S.A.).

### High-performance Liquid Chromatography Analysis Method and Calculation Method for Content Ratio of Cellulose

A guard column (KS-G manufactured by Showa Denko K.K.) and a separation column (KS-802 manufactured by Showa Denko K.K.) were connected to each other, and the column temperature was set to 75° C. Pure water was supplied as an eluting solution at a rate of 0.5 ml/min, and a separated component was subjected to quantitative determination with a differential refractive index detector. Thus, the concentrations of glucose and xylose were determined, and the content ratios of cellulose, hemicellulose, and holocellulose were calculated based on the following equation.

Content ratio of cellulose (%)={mass of filtrate (g)×(concentration of glucose (%)×0.9}/mass of weighed biomass (g)×100

(The numerical value “0.9” in the equation is a coefficient for correcting changes in molecular weight caused by hydrolysis of cellulose.)

### Evaluation of Saccharification of the Hydrothermally-Treated Sample

### Measurement of a Solid Content Concentration

2 to 3 g of a sample of the sample subjected to hydrothermal treatment was put on a Kett-type moisture tester and dried to calculate the residue on evaporation, and the ratio of the residue was defined as the solid content concentration.

### Preparation of Enzyme Solution

4.3 g of Meicelase (trademark, cellulase manufactured by Meiji Seika Kaisha, Ltd. (currently Meiji Seika Pharma Co., Ltd.)) was dissolved in 96.7 g of pure water.

The FPU activity (Filter Paper Assay for Saccharifying Cellulase) of the enzyme solution was 15 FPU/g, which was determined according to an analysis method of International Union of Pure and Applied Chemistry (IUPAC) (Pure & Appl. Chem., Vol. 59, No. 2, pp. 257-268, 1987).

### Saccharification Reaction

A rotor was put in a 50 ml glass vessel with a cover, and a composition subjected to pretreatment was weighed so that the amount of the solid content was 1.5 g. Then, pure water was added thereto to give a total of 8.0 g. The pH of the resultant was adjusted to pH 5 by adding a 2.5 M NaOH solution while being mixed with a spatula. The pH was measured by putting the sample on a pH meter (type: P-212) manufactured by Horiba, Ltd., and after the measurement, the sample was returned to a glass container.

After the adjustment of pH, 1.0 g of an enzyme solution was added to the sample while being mixed with a spatula. Further, pure water was added thereto to give a total of 10 g while washing the spatula. Then, the glass container was covered with a lid and the content was immediately subjected to a saccharification reaction with the enzyme in a thermostat bath at 40° C. for 72 hours (Hr) while being stirred. The resultant saccharified solution was subjected to quantitative determination for soluble sugar (the total of glucose, cellobiose, cellotriose, cellotetraose, cellopentaose, cellohexaose, arabinose, xylose, xylobiose and xylotriose) and furfural by high-performance liquid chromatography analysis. Thus, a utilization rate of soluble sugar were calculated by the following equations.

Saccharification rate (%)={concentration of glucose (%)/(concentration of solid content of a sample in a saccharification reaction solution (%)/100)}×(cellulose content of the raw material biomass (%)/100)}×0.9×100

### Evaluation of the Culture of a Saccharification Solution

### Seed Culture

The NBRC2346 strain of Saccharomyces. cerevisiae as being a standard strain of yeast was cultured at 30° C. for one night in a potato dextrose agar (PDA) medium.

### Preculture

An aqueous medium solution was prepared to contain 6.7 g/l of yeast nitrogen base without amino acid (manufactured by DIFCO, hereinafter abbreviated as “YNB”) and 20 g/l of reagent glucose and sterilized (hereinafter referred to as “SD glucose medium”). 4 ml of the medium solution was put in a test tube having a diameter of 18 mm under aseptic conditions, and a loopful of the seed-cultured strain was inoculated and cultured at 30° C. and a rotation number of 300 rpm for one night.

### Main Culture

A culture solution was adjusted to have a final concentration of 20 g/l of glucose and 6.7 g/l of YNB by adding YNB and water to a supernatant obtained by centrifuging each of the saccharification solutions. 4 ml of a filtrate obtained by sterilization filtration was put in a medium solution in a sterilized test tube under aseptic conditions, and 40 μl of the preculture solution (inoculation ratio: 1%) was inoculated thereto, and cultured at 30° C. and a rotation number of 300 rpm for one night.

As a control sample, the culture was conducted in a SD medium as well.

### Analysis of Ethanol

A sample for analyzing ethanol was prepared by diluting with water a supernatant obtained by centrifuging the main culture solution so as to have an ethanol concentration of 0.1 to 0.5 percent by volume. The sample was subjected to gas chromatography analysis by using GC-18A manufactured by Shimadzu Corporation as a device and Thermon-300 5% (inner diameter: 3 mm, length: 3m) as a column, under the following conditions to determine the ethanol concentration in the sample:
carrier gas: helium gas, 50 ml/min.,
injection amount: 1 μm,
rate of temperature increase: after the temperature was raised from 50° C. to 100° C. at an increase rate of 20° C./minute, the temperature was maintained at 100° C.,
temperature of a detector: 250° C.

### Example 1

The 3-mm bagasse was loaded into the screw extruder B having a screw rotation number of 350 rpm with a mass feeder and a compactor at a feed rate of 5.0 kg/Hr of a mass of the bagasse as it was and 4.5 kg/Hr in terms of dry mass. In the pulverization section, water was added through a liquid feed line at a feed rate of 4.8 kg/Hr, and a water content ratio was adjusted so that a water content ratio of 54 mass % (solid content concentration: 46 mass %) was continuously obtained in the pulverization section upstream of the heating section. The raw material was subjected to hydrothermal treatment in the heating section so that the temperature of the raw material in the heating section (hereinafter abbreviated as “hydrothermal temperature”) and a pressure in the heating section were set to 175° C. and 5 MPa, respectively, and then cooled to 70° C. or less in the cooling section with a water cooling jacket, followed by recovery of a sample from a discharge port. A time of passage through the heating section (hereinafter abbreviated as “hydrothermal time”) under such conditions was 7.5 minutes.

The recovered sample was evaluated for the saccharification, and further the result of the culture of the saccharification solution was evaluated.

### Examples 2 to 4 and Comparative Examples 1 to 4

The saccharification and culture were conducted in the same manner as in Comparative Example 1 except that the conditions were changed as shown in Table 2 to evaluate the results.

TABLE 2

| | | | | | | Number of |
|---|---|---|---|---|---|---|
| | | Feed rate | | | | seal ring |
| | | | In terms | Screw | | elements |
| | | Mass as | of dry | rotation | | in the |
| | Kind of | it is | mass | number | Screw | heating |
| | bagasse | [kg/Hr] | [kg/Hr] | [rpm] | extruder | section |
| Example 1 | 3-mm | 5.0 | 4.5 | 350 | B | 5 |
| | bagasse | | | | | |
| Comparative | 3-mm | 2.5 | 2.3 | 350 | E | 0 |
| Example 1 | bagasse | | | | | |
| Example 2 | 3-mm | 5.0 | 4.5 | 350 | A | 6 |
| | bagasse | | | | | |
| Comparative | 3-mm | 2.5 | 2.3 | 350 | D | 0 |
| Example 2 | bagasse | | | | | |
| Example 3 | Untreated | 20.0 | 10.0 | 200 | C | 4 |
| | bagasse | | | | | |
| Comparative | Untreated | 20.0 | 10.0 | 200 | C | 4 |
| Example 3 | bagasse | | | | | |
| Example 4 | Untreated | 40.0 | 20.0 | 200 | C | 4 |
| | bagasse | | | | | |
| Comparative | Untreated | 40.0 | 20.0 | 200 | C | 4 |
| Example 4 | bagasse | | | | | |

| | | Water | | | | |
|---|---|---|---|---|---|---|
| | | content | Maximum | | | |
| | Water | ratio in | grain size | | | |
| | feed rate | pulverization | immediately | | | |
| | through | section | upstream of | | | Hydro- |
| | liquid | (solid content | heating | Temper- | | thermal |
| | feed line | concentration) | section | ature | Pressure | time |
| | [kg/Hr] | [mass %] | [μm] | [° C.] | [MPa] | [min] |
| Example 1 | 4.8 | 54 (46) | <1000 | 175 | 5 | 7.5 |
| Comparative | 2.9 | 57 (43) | <1000 | 175 | 5 | 7.5 |
| Example 1 | | | | | | |
| Example 2 | 3.6 | 48 (52) | <1000 | 190 | 5 | 7.5 |
| Comparative | 2.9 | 57 (43) | <1000 | 190 | 5 | 7.5 |
| Example 2 | | | | | | |
| Example 3 | 5.0 | 60 (40) | <1000 | 189 | 5 | 2.5 |
| Comparative | 5.0 | 60 (40) | <1000 | 210 | 5 | 2.5 |
| Example 3 | | | | | | |
| Example 4 | 10.0 | 60 (40) | <1000 | 210 | 5 | 1.2 |
| Comparative | 10.0 | 60 (40) | <1000 | 220 | 5 | 1.2 |
| Example 4 | | | | | | |

Table 3 shows the results of the saccharification (the furfural concentration after the saccharification, the cellulose concentration before the saccharification, the glucose concentration after the saccharification, the utilization rate of glucose, and the furfural ratio with respect to glucose after the saccharification) of the treated samples in Examples 1 to 4 and Comparative Examples 1 to 4.

TABLE 3

| | Concentration | Concentration | Concentration | | Furfural ratio |
|---|---|---|---|---|---|
| | of furfural | of cellulose | of glucose | Glucose | with respect to |
| | after the sacchar- | before the sacchar- | after the sacchar- | utilization | glucose after the |
| | ification (ppm) | ification (%) | ification (%) | rate (%) | saccharification |
| Example 1 | 594 | 6.3% | 3.8% | 54% | 1.6% |
| Comparative | 560 | 6.3% | 1.2% | 17% | 4.7% |
| Example 1 | | | | | |
| Example 2 | 1231 | 6.3% | 4.0% | 57% | 3.1% |
| Comparative | 1103 | 6.3% | 1.3% | 19% | 8.5% |
| Example 2 | | | | | |
| Example 3 | 650 | 5.7% | 3.2% | 51% | 2.0% |
| Comparative | 1894 | 5.7% | 3.4% | 54% | 5.6% |
| Example 3 | | | | | |
| Example 4 | 689 | 5.7% | 3.3% | 52% | 2.1% |
| Comparative | 1975 | 5.7% | 3.4% | 54% | 5.8% |
| Example 4 | | | | | |

### Comparison of Saccharification Performance of the Hydrothermally-Treated Sample Depending on Conditions of Extruders

The hydrothermal treatment was performed under the same hydrothermal conditions at a temperature of 170° C. and in time of 7.5 minutes in Examples 1 and Comparative Example 1, and at a temperature of 190° C. and in time of 7.5 minutes in Example 2 and Comparative Example 2, respectively, with an extruder having a different screw configuration.

The extrusion hydrothermal treatment in Examples 1 and 2 was performed with screw extruder B (FIG. 1(B)) in which 5 sets of seal ring elements were arranged and with screw extruder A (FIG. 1(A)) in which 6 sets of elements are arranged. In contrast, in Comparative Examples 1 and 2, the extrusion hydrothermal treatment was performed with the screw extruders E and D (FIG. 1(E) and FIG. 1(D)) in which the seal ring element was not arranged.

The glucose concentration and glucose utilization rate of the treated sample under the hydrothermal treatment conditions at 175° C. in 7.5 minutes were 3.8% and 54%, respectively, in Example 1 including 5 sets of the seal ring elements, while 1.2% and 17% in Comparative Example 1 including 0 set of the seal ring element. The glucose concentration and glucose utilization rate of the treated sample under the hydrothermal treatment conditions at 190° C. in 7.5 minutes were 4.0% and 57% in Example 2 including 6 sets of the seal ring elements, while 1.3% and 19% in Comparative Example 1 including 0 set of the seal ring element, and it was confirmed that the saccharification performance of the sample significantly reduces when no seal ring element is arranged.

Such effect exhibited by the seal ring element is presumed to be caused as follows: high grinding stress is applied to the cellulose-containing biomass serving as a raw material when it passes through an extremely narrow clearance portion between the seal ring and the cylinder concurrently with the hydrothermal treatment, and thus the saccharification performance of cellulose in the biomass is improved.

From the above-mentioned results, it was confirmed that as device conditions of a screw extruder for attaining high saccharification performance, arrangement of a plurality of seal ring elements in the heating section was effective.

### Coarse Pulverization in the Pulverization Section before the Hydrothermal Treatment

Examples and Comparative Examples in this description were test examples in which the hydrothermal treatment was able to be performed with a screw extruder. The maximum grain sizes before the extrusion hydrothermal treatment were each determined through microscopic observation of the sample extracted from the pulverization section immediately upstream of the heating section. The maximum grain sizes of the samples were found to be each 1,000 μm or less.

Meanwhile, a test was performed, but the hydrothermal treatment was not able to be actually performed under the following conditions: i.e. the hydrothermal treatment with a screw extruder having a screw configuration in which the seal ring was removed from the seal ring elements in each of the pulverization sections of the screw extruders A and E. The hydrothermal treatment was attempted by supplying 3-mm bagasse to the screw extruders, but the pressure of vapor was not able to be maintained on an upstream side, and a backward flow of the vapor was generated. The treatment failed. In those cases, the maximum grain sizes of the raw material collected from the pulverization section before the hydrothermal treatment each exceeded 1,000 μm, and the pulverization was not sufficient.

From the above-mentioned results, it is presumed that, in the extrusion hydrothermal treatment, the coarse pulverization in the pulverization section exhibits an effect of improving the saccharification performance and an effect of sealing vapor to be generated in the hydrothermal section on an upstream side. It was confirmed that the coarse pulverization was preferably performed to such degree that the maximum grain size fell below 1,000 μm.

### Operation Results in Terms of Conditions of Cooling Section

Examples and Comparative Examples in this description were test examples in which the hydrothermal treatment was able to be performed with a screw extruder. In each screw extruder, a cooling system of a water cooling jacket and a liquid feed line was used in the cooling section, and a pressure regulating valve was mounted to a discharge port. In the test examples, the temperature in the cooling section was reduced to 100° C. or less through cooling with a water cooling jacket in Examples 1 to 3 and Comparative Examples 1 to 3, and through cooling with the water cooling jacket and water feeding from a liquid feed line to the biomass to be treated in combination in Examples 4 and Comparative Example 4. The hydrothermal treatment was able to be stably and continuously performed in all the test examples.

Meanwhile, a test was performed, but the hydrothermal treatment was not able to be actually performed under the following conditions. When the treatment was performed without using any cooling system of a water cooling jacket and a liquid feed line in each screw extruder, the temperature in the cooling section kept a high temperature state exceeding 100° C., a sample intermittently jetted together with vapor, and the treatment was not able to be stably performed in the case of each screw extruder. As described above, it was confirmed that, for stable operation, the temperature in the cooling section needed to be reduced to 100° C. or less through use of the cooling system of a water cooling jacket and/or a liquid feed line.

### Saccharification Performance of the Hydrothermally-Treated Sample and Furfural Concentration

The saccharification performance of the treated samples under different hydrothermal treatment conditions were compared based on the results of the hydrothermal treatment with a screw extruder in which seal ring elements are arranged in the heating section to ensure the improvement of the saccharification performance in Examples 1 to 4 and Comparative Examples 3 to 4.

If the grain size and the water content of the bagasse are adjusted to the same level by pulverization and water feeding in the pulverization section, and other device conditions and treatment conditions are the same, it can be said that saccharification performance is unaffected due to the difference between “3-mm bagasse” which is dried and coarsely-pulverized in advance and “untreated bagasse”. The difference in the feed rate of bagasse affects the retention time of the raw material in the device and is associated with the hydrothermal time. Therefore, the feed rate can be an operating factor to set the hydrothermal time: for example, the rate is increased to shorten the hydrothermal time and is decreased to lengthen the hydrothermal time.

The results of the evaluation of the saccharification performance of each of the treated samples showed glucose concentration of 3% or higher and glucose utilization rate of 51% or higher under any hydrothermal conditions in the examples, and it was confirmed that good saccharification performance can be attained by the hydrothermal treatment when the temperature and time fall within the ranges of 175° C. to 220° C. and 1 to 10 minutes, respectively.

The furfural concentration was 594 ppm in the sample treated at 175° C. in 7.5 minutes in Example 1, 1231 ppm in the sample treated at 190° C. in 77.5 minutes in Example 2, 650 ppm in the sample treated at 190° C. in 2.5 minutes in Example 3, 1894 ppm in the sample treated at 210° C. in 2.5 minutes in Comparative Example 3, 689 in the sample treated at 210° C. in 1.2 minutes in Example 4, and 1975 ppm in the sample treated at 220° C. in 1.2 minutes in Comparative Example 4. Thus, the concentration had a tendency to increase under hydrothermal conditions at a high temperature or for a long period of time.

From the above-mentioned saccharification results, it was confirmed that the conditions to attain both of good saccharification performance and the concentration lower than 1,000 ppm were roughly 2.5 minutes at a temperature 200° C. or lower and 1.2 minutes at a temperature exceeding 200° C.

It should be noted that the furfural concentration in the saccharification solution of the present experiment directly reflects the generation behavior of furfural as a by-product at the time of hydrothermal treatment because the enzyme reaction is directly conducted without washing the thermally-treated product, and therefore can be a value to determine good or bad hydrothermal conditions.

### Evaluation of the Culture Results

A medium was adjusted to have a final concentration of glucose of 2% and the sample after the saccharification was cultured in the medium with the standard strain of yeast and was evaluated for the ethanol concentration after 48 hours (Hr). In the evaluation of the culture results, the culture was conducted using a glucose reagent as well as a control sample.

Table 3 shows the results of the culture (glucose concentration in the medium, ethanol concentration, and ethanol concentration ratio with respect to the control) and the furfural concentration.

TABLE 4

| | Glucose | Furfural | | Furfural ratio | |
|---|---|---|---|---|---|
| | concen- | concen- | Ethanol | with respect to | |
| | tration | tration | concen- | glucose after the | |
| | in the | in the | tration | saccharification | |
| | medium (%) | medium | (wt/V %) | (%) | |
| Example 1 | 2.0% | 313 ppm | 0.78% | 105% | ∘ |
| Comparative | — | — | — | — | — |
| Example 1 | | | | | |
| Example 2 | 2.0% | 616 ppm | 0.77% | 104% | ∘ |
| Comparative | — | — | — | — | — |
| Example 2 | | | | | |
| Example 3 | 2.0% | 406 ppm | 0.77% | 104% | ∘ |
| Comparative | 2.0% | 1114 ppm | 0.58% | 78% | x |
| Example 3 | | | | | |
| Example 4 | 2.0% | 418 ppm | 0.75% | 101% | ∘ |
| Comparative | 2.0% | 1162 ppm | 0.50% | 68% | x |
| Example 4 | | | | | |
| Control | 2.0% | 0 ppm | 0.74% | 100% | |

While the ethanol concentration of the control obtained by the culture using a glucose reagent was 0.74%, the ethanol concentration and the ratio to the concentration of the control were 0.78% and 105% in the sample treated at 175° C. in 7.5 minutes in Example 1, 0.77% and 104% in the sample treated at 190° C. in 77.5 minutes in Example 2, 0.77% and 104% in the sample treated at 190° C. in 2.5 minutes in Example 3, 0.58% and 78% in the sample treated at 210° C. in 2.5 minutes in Comparative Example 3, 0.75% and 101% in the sample treated at 210° C. in 1.2 minutes in Example 4, and 0.50% and 68% in the sample treated at 220° C. in 1.2 minutes in Comparative Example 4. Thus, in Comparative Examples 3 and 4 having a high furfural concentration, the ethanol concentration was lower than that of the control and it was confirmed that the fermentation was inhibited.

Although Example 2 had a high furfural concentration exceeding 1,000 ppm after the saccharification reaction, the glucose concentration was also high unlike in the Comparative Examples, resulting in a low furfural ratio with respect to glucose. Therefore, the furfural concentration in the medium adjusted to have a glucose concentration of 2% was suppressed to 616 ppm in contrast to the concentration exceeding 1,000 ppm in Comparative Examples 3 and 4, and a good ethanol concentration was attained.

From the foregoing results of the saccharification, it was confirmed that the conditions to obtain good saccharification performance without inhibiting fermentation were roughly 2.5 minutes or less at a temperature 200° C. or lower and 1.2 minutes or less at a temperature exceeding 200° C., as with the conditions to make the furfural concentration lower than 1,000 ppm.

### INDUSTRIAL APPLICABILITY

According to the pretreatment method for cellulose-containing biomass of the present invention conducting kneading pulverization having a grinding effect concurrently with hydrothermal treatment with a screw extruder, the generation of furfural as a by-product can be suppressed at the time of saccharification of a cellulose-containing biomass composition to be obtained, and an industrially useful composition exhibiting high saccharification performance in saccharification of cellulose-containing biomass can be efficiently obtained.

1. A treatment method for biomass, which is a pretreatment method comprising continuously performing hydrothermal treatment through use of a screw extruder to produce from cellulose-containing biomass serving as a raw material a biomass composition for saccharification containing only a small quantity of furfural as being a fermentation-inhibiting component,
the treatment method sequentially comprising:
pulverizing, in a pulverization section of the screw extruder, the cellulose-containing biomass serving as a raw material so as to have a maximum grain size of 1,000 μm or less and adjusting a water content ratio thereof to from 30% to 80%;
performing hydrothermal treatment in a heating section of the screw extruder, in which an element including a seal ring and at least one set of a kneading disc and/or a left-hand screw is arranged immediately upstream of the seal ring, at a temperature of from 150° C. to 200° C. for 0.1 minute to 15 minutes while performing kneading pulverization having a grinding effect; and
cooling, in a cooling section of the screw extruder downstream of the heating section, a treated product to 100° C. or less to recover the treated product of the cellulose-containing biomass.

2. A treatment method for biomass, which is a pretreatment method comprising continuously performing hydrothermal treatment through use of a screw extruder to produce from cellulose-containing biomass serving as a raw material a biomass composition for saccharification containing only a small quantity of furfural as being a fermentation-inhibiting component,
the treatment method sequentially comprising:
pulverizing, in a pulverization section of the screw extruder, the cellulose-containing biomass serving as a raw material so as to have a maximum grain size of 1,000 μm or less and adjusting a water content ratio thereof to from 30% to 80%;
performing hydrothermal treatment in a heating section of the screw extruder, in which an element including a seal ring and at least one set of a kneading disc and/or a left-hand screw is arranged immediately upstream of the seal ring, at a temperature of from 200° C. to 215° C. for 0.1 minute to 2.0 minutes while performing kneading pulverization having a grinding effect; and
cooling, in a cooling section of the screw extruder downstream of the heating section, a treated product to 100° C. or less to recover the treated product of the cellulose-containing biomass.

3. The treatment method for biomass as claimed in claim 1, in which, in the cooling section downstream of the heating section, the cooling is performed by installing a water cooling jacket and/or a liquid feed line.

4. The treatment method for biomass as claimed in claim 1, in which the screw extruder is a co-rotating twin-screw extruder.

5. The treatment method for biomass as claimed in claim 1, in which the cellulose-containing biomass is soft biomass.

6. A production method for a biomass composition for saccharification, comprising conducting the treatment method as claimed in claim 1.

7. A production method for a sugar, comprising hydrolyzing a biomass composition obtained by the production method as claimed in claim 6.
